(19) United States
(12) Patent Application Publication
(10) Pub. No.: US 2011/0162981 A1
(43) Pub. Date: Jul. 7, 2011

(54) METHODS AND COMPOSITIONS FOR PREVENTING, REDUCTING OR TREATING DAMAGE CAUSED BY ISCHEMIA AND SCHEMIA-LIKE CONDITIONS

(76) Inventors: Yuanlong Pan, Chesterfield, MO (US); Olivier Ballevre, Beijing (CN); Peter Bucheli, Bremgarten (CH)

(21) Appl. No.: 12/737,602

(22) PCT Filed: Jul. 30, 2009
(86) PCT No.: PCT/US2009/004414

§ 371 Date: Mar. 18, 2011

Related U.S. Application Data
(60) Provisional application No. 61/137,381, filed on Jul. 30, 2008.

(51) Int. Cl. B65D 71/00; A61K 36/815; A61P 25/28

(52) U.S. Cl. 206/223; 424/777

(57) ABSTRACT

Methods for using wolfberry for one or more of preventing, reducing, or treating damage caused by ischemia and ischemia-like conditions; preventing, reducing, or treating strokes and damage caused by strokes; preventing, reducing, or treating alterations in brain function; preventing, reducing, or treating cognitive dysfunction syndrome; preventing, reducing, or treating mild cognitive impairment; preventing, reducing, or treating dementia; preventing, reducing, or treating a decline in social interaction; preventing, reducing, or treating age-related behavioral changes; facilitating, maintaining, and enhancing cognitive function; facilitating, maintaining, and enhancing learning and memory; facilitating, maintaining, and enhancing optimal brain function; reducing memory loss; increasing trainability; improving the quality of life; and promoting the health and wellness in an animal.

### CROSS REFERENCE TO RELATED APPLICATIONS

This application is a national stage application under 35 U.S.C. §371 of PCT/US2009/004414 filed Jul. 30, 2009, which claims priority to U.S. Provisional Application Ser. No. 61/137,381 filed Jul. 30, 2008, the disclosures of which are incorporated herein by reference.

### BACKGROUND OF THE INVENTION

1. Field of the Invention

The invention relates generally to methods and compositions for preventing, reducing, or treating damage caused by ischemia and ischemia-like conditions and particularly to methods and compositions that use wolfberry for preventing, reducing, or treating damage caused by ischemia and ischemia-like conditions in animals.

2. Description of Related Art

Wolfberry is a fruit from the family Solanaceae in the species Lycium barbarum or Lycium chinense. Wolfberry is known by several names, e.g., goji berry. Wolfberry is known as an agent in Chinese medicine and is claimed to be useful for many purposes, e.g., enhancing the immune system, improving vision, protecting the liver, improving circulation, and boosting sperm production. However, there are few, if any, scientific studies that validate these claims. US Patent Application 20060088643 discloses that wolfberry compositions, wolfberry extracts, and the use of wolfberry to strengthen muscles and bone, protect liver function, replenish the vital essence, treat diabetes, prevent aging, and improve visual acuity. US200710212433 discloses a method of improving absorption of an antioxidant by co-administering at least one antioxidant with an effective amount of a Gac Fruit extract, e.g., wolfberry. WO05018656A1 discloses using wolfberry extract to prevent brain nerve cell death and Alzheimer's disease. US20050238654 discloses that wolfberry can be used to lower blood pressure and cholesterol levels, acting mainly on the liver and kidney. US20050281902 discloses a method for inhibiting metastasis of colon, lung, liver, kidney, breast, or cervical cancer using Lycium barbarum extract. The reference also discloses that Lycium barbarum helps improve vision and prevent headaches and dizziness caused by liver and kidney deficiencies and may be effective in treating mild diabetes.

Ischemia is the inadequate flow of blood to a part of the body caused by constriction, rupture, or blockage of blood vessels. Loss of oxygen due to the insufficient blood supply causes tissue to become hypoxic or anoxic and often results in tissue damage or dysfunction, e.g., necrosis and apoptosis. Ischemia can be caused by injury or disease, e.g., atherosclerosis, hypotension, embolism, g-forces, sickle cell disease, thromboembolism, and stroke. Strokes include three major types, i.e., ischemic strokes, hemorrhagic strokes, and transient ischemic attacks. Ischemic strokes occur when a blood vessel supplying blood to the brain is blocked. Hemorrhagic strokes occur when a blood vessel supplying blood to the brain ruptures. Hemorrhagic strokes can be classified as intracerebral or subarachnoid depending on whether the blood goes into or around the brain. Transient ischemic attacks are minor or warning strokes, typically lasting less than one hour. Strokes also cause brain damage, impair cognitive function, impede learning, impede memory, reduce social interaction, decrease trainability, reduce optimal brain function, cause memory loss, and increase brain aging. Ischemia-like conditions occur when there is not enough oxygen in the blood to supply needed oxygen to tissue, e.g., following hemorrhages or low levels of oxygen in the blood.

Ischemia often results from strokes, including transient ischemic attacks that produce stroke-like symptoms but no lasting tissue damage. However, strokes can cause sudden headaches, numbness, weakness, dizziness, confusion, loss of balance, loss of coordination and difficulty speaking, understanding, seeing, or walking. The short duration of these symptoms and lack of permanent brain damage is the main difference between transient ischemic attacks and other strokes. However, transient ischemic attacks are an important indicator of future stroke because the cause of transient ischemic attacks is often the cause of other strokes. Transient ischemic attacks are known to increase as an animal ages and be responsible for various age-related behavioral changes.

Ischemia, from strokes or otherwise, causes many adverse effects in an animal, particularly to the animal brain. For example, ischemia may be responsible for altering brain functions such as cognitive function (e.g., enhancing cognitive function), sensory function (sense of smell), auditory system (sense of hearing), visual perception, motion perception, depth perception, somatosensory function (e.g., pressure, temperature, pain, posture, visceral sense, and facial expression), motor function, language processing, speech production and comprehension, and sign production and comprehension. Changes in cognitive function include functions such as learning, attention, memory, concentrating, planning, logic thinking, reasoning, decision-making, spatial orientation, communicating, following instructions, carrying out daily activities, and the like. Similarly, ischemia may cause “cognitive dysfunction syndrome” and related symptoms such as disorientation (e.g., confusion, wandering aimlessly, going to wrong side of doors, appearing to forget previously learned tasks, getting “stuck” in corners and behind furniture), abnormal interaction with others and the environment, sleep/awake pattern disruption (e.g., sleep more during the day, sleeping much more deeply, and awake at times during the night), loss of skills, and loss of house training (e.g., inappropriate urination and defecation). Further, ischemia, particularly from transient ischemic attacks, may cause “mild cognitive impairment” and resulting symptoms such as problems with memory, language, or other mental functions severe enough to be noticeable but not serious enough to interfere with daily life. In addition, ischemia can cause dementia, including vascular dementia, by significantly impairing intellectual functioning such that it interferes with normal activities and relationships, generally by causing a loss of neurons. Vascular dementia is typically caused by blockage or rupture of a big blood vessel or a series of small blood vessels. Such blockage or rupture results in ischemia by disrupting blood supply to a large area of the brain. Vascular dementia may also develop after a series of small strokes disrupt blood supply to various brain regions.

Ischemia is adverse to the health and wellness of an animal and results in a lower quality of life for the animal. There is, therefore, a need for methods and compositions useful for promoting the health and wellness and improving the quality of life for animals by preventing, reducing, or treating damage caused by ischemia and ischemia-like conditions in an animal, particularly ischemia caused by strokes.

### SUMMARY OF THE INVENTION

It is, therefore, an object of the invention to provide methods and compositions useful for one or more of preventing, reducing, or treating damage caused by ischemia and ischemia-like conditions; preventing, reducing, or treating strokes and damage caused by strokes; preventing, reducing, or treating alterations in brain function; preventing, reducing, or treating cognitive dysfunction syndrome; preventing, reducing, or treating mild cognitive impairment; preventing, reducing, or treating dementia; preventing, reducing, or treating a decline in social interaction; preventing, reducing, or treating age-related behavioral changes; facilitating, maintaining, or enhancing cognitive function; facilitating, maintaining, or enhancing learning and memory; facilitating, maintaining, or enhancing optimal brain function; reducing memory loss; increasing trainability; improving the quality of life; and promoting the health and wellness in an animal.

One or more of these or other objects individually or collectively are achieved using novel methods and compositions useful for counteracting the adverse affects of ischemia and ischemia-like conditions in an animal. The compositions comprise wolfberry and the methods comprise administering wolfberry to an animal in an amount effective for one or more of preventing, reducing, or treating damage caused by ischemia and ischemia-like conditions; preventing, reducing, or treating strokes and damage caused by strokes; preventing, reducing, or treating alterations in brain function; preventing, reducing, or treating cognitive dysfunction syndrome; preventing, reducing, or treating mild cognitive impairment; preventing, reducing, or treating dementia; preventing, reducing, or treating a decline in social interaction; preventing, reducing, or treating age-related behavioral changes; facilitating, maintaining, or enhancing cognitive function; facilitating, maintaining, or enhancing learning and memory; facilitating, maintaining, or enhancing optimal brain function; reducing memory loss; increasing trainability; improving the quality of life; and promoting the health and wellness in an animal.

Other and further objects, features, and advantages of the invention will be readily apparent to those skilled in the art.

### DETAILED DESCRIPTION OF THE INVENTION

### Definitions

The term “wolfberry” means one or more fruits from the family Solanaceae in the species Lycium barbarum or Lycium chinense and extracts or portions thereof.

The term “animal” means any animal that could benefit from the methods or compositions of the invention, particularly an animal susceptible to or suffering from injury or damage caused by ischemia and ischemia-like conditions that result in decreased blood flow to tissue, e.g., including low oxygen pressure, low nutrient levels, the accumulation of cellular products, vascular aging, and breathing difficulty. Generally, the animal is a human, avian, bovine, canine, equine, feline, hicrine, lupine, murine, ovine, or porcine animal.

The term “companion animal” means domesticated animals such as cats, dogs, rabbits, guinea pigs, ferrets, hamsters, mice, gerbils, horses, cows, goats, sheep, donkeys, pigs, and the like.

The term “effective amount” means an amount of a compound, composition, medicament, or other material that is effective to achieve a particular physiological or biological result. For the invention, such results include, but are not limited to, one or more of preventing, reducing, or treating damage caused by ischemia and ischemia-like conditions; preventing, reducing, or treating strokes and damage caused by strokes; preventing, reducing, or treating alterations in brain function; preventing, reducing, or treating cognitive dysfunction syndrome; preventing, reducing, or treating mild cognitive impairment; preventing, reducing, or treating dementia; preventing, reducing, or treating a decline in social interaction; preventing, reducing, or treating age-related behavioral changes; facilitating, maintaining, or enhancing cognitive function; facilitating, maintaining, or enhancing learning and memory; facilitating, maintaining, or enhancing optimal brain function; reducing memory loss; increasing trainability; improving the quality of life; and promoting the health and wellness in an animal.

The term “dietary supplement” means a product that is intended to be ingested in addition to a normal animal diet. Dietary supplements may be in any form, e.g., solid, liquid, gel, tablet, capsule, powder, and the like. Preferably they are provided in convenient dosage forms, e.g., in sachets. Dietary supplements can be provided in bulk consumer packages such as bulk powders, liquids, gels, or oils. Similarly such supplements can be provided in bulk quantities to be included in other food items such as snacks, treats, supplement bars, beverages, and the like.

The term “aging” means being of an advanced age such that an animal has reached or exceeded 50% of the average life expectancy for the animal's species and/or breed within such species. For example, if the average life expectancy for a given breed of dog is 12 years, then an “aging animal” within that breed is 6 years old or older.

The term “food” or “food product” or “food composition” means a product or composition that is intended for ingestion by an animal, including a human, and provides nutrition to the animal.

The term “extending the prime” means extending the number of years an animal lives a healthy life and not just extending the number of years an animal lives, e.g., an animal would be healthy in the prime of its life for a relatively longer time.

The term “regular basis” means at least monthly dosing with wolfberry and more preferably weekly dosing. More frequent dosing or consumption, such as twice or three times weekly, is preferred in certain embodiments. Still more preferred are regimens that comprise at least once daily consumption, e.g., when wolfberry is a component of a food composition that is consumed at least once daily.

The term “single package” means that the components of a kit are physically associated in or with one or more containers and considered a unit for manufacture, distribution, sale, or use. Containers include, but are not limited to, bags, boxes, cartons, bottles, packages such as shrink wrap packages, stapled or otherwise affixed components, or combinations thereof. A single package may be containers of individual wolfberry and food compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag or other container containing one component and directions instructing the user to go to a website, contact a recorded message or a fax-back service, view a visual message, or contact a caregiver or instructor to obtain instructions on how to use the kit or safety or technical information about one or more components of a kit.

The dosages expressed herein are in milligrams per kilogram of body weight per day (mg/kg/day) unless expressed otherwise.

All percentages expressed herein are by weight of the composition on a dry matter basis unless specifically stated otherwise. The skilled artisan will appreciate that the term “dry matter basis” means that an ingredient's concentration or percentage in a composition is measured or determined after any free moisture in the composition has been removed.

As used herein, ranges are in shorthand, so as to avoid having to list and describe each and every value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a supplement”, “a method”, or “a food” includes a plurality of such “supplements”, “methods”, or “foods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Similarly, the term “examples,” particularly when followed by a listing of terms, is merely exemplary and illustrative and should not be deemed to be exclusive or comprehensive.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the invention, the preferred compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### The Invention

In one aspect, the invention provides methods for preventing, reducing, or treating damage caused by ischemia and ischemia-like conditions in an animal. The methods comprise administering wolfberry to the animal in an amount effective for preventing, reducing, or treating damage caused by ischemia and ischemia-like conditions in the animal. The methods are effective for preventing, reducing, or treating damage to any body tissue, preferably the brain.

In another aspect, the invention provides methods for preventing, reducing, or treating strokes and damage caused by strokes in an animal. The methods comprise administering wolfberry to the animal in an amount effective for preventing, reducing, or treating strokes and damage caused by strokes in the animal. The methods are effective for preventing, reducing, or treating strokes because the methods combat ischemia-like conditions caused by aging and disease, particularly strokes caused by damage from previous transient ischemic attacks.

In a further aspect, the invention provides methods for preventing, reducing, or treating alterations in brain function in an animal. The methods comprise administering wolfberry to the animal in an amount effective for preventing, reducing, or treating alterations in brain function in the animal. The invention is useful for preventing, reducing, or treating any alteration in brain function related to ischemia or ischemia-like conditions including, but not limited to, cognitive functions, sensory functions, auditory functions, visual functions, motion perception, depth perception, somatosensory functions, motor functions, language processing, speech production and comprehension, and sign production and comprehension.

In one aspect, the invention provides methods for preventing, reducing, or treating cognitive dysfunction syndrome in an animal. The methods comprise administering wolfberry to the animal in an amount effective for preventing, reducing, or treating cognitive dysfunction syndrome in the animal. The invention is useful for preventing, reducing, or treating disorientation, abnormal interaction with others, abnormal interaction with the environment, sleep/awake pattern disruption, loss of skills, and loss of house training.

In one aspect, the invention provides methods for preventing, reducing, or treating mild cognitive impairment in an animal. The methods comprise administering wolfberry to the animal in an amount effective for preventing, reducing, or treating mild cognitive impairment in the animal. The invention is useful for preventing, reducing, or treating problems with memory, problems with language, or other mental functions severe enough to be noticeable but not serious enough to interfere with daily life.

In another aspect, the invention provides methods for preventing, reducing, or treating dementia in an animal. The methods comprise administering wolfberry to the animal in an amount effective for preventing, reducing, or treating dementia in the animal. The invention is useful for preventing, reducing, or treating dementia that results from any ischemia or ischemia-like condition, particularly vascular dementia.

In one aspect, the invention provides methods for preventing, reducing, or treating a decline in social interaction in an animal. The methods comprise administering wolfberry to the animal in an amount effective for preventing, reducing, or treating a decline in social interaction in the animal. The invention is useful for preventing, reducing, or treating a decline in social interaction that results from any ischemia or ischemia-like condition, particularly a decline in social interaction in an aging animal.

In one aspect, the invention provides methods for preventing, reducing, or treating age-related behavioral changes in an animal. The methods comprise administering wolfberry to the animal in an amount effective for preventing, reducing, or treating age-related behavioral changes in the animal. The invention is useful for preventing, reducing, or treating age-related behavioral changes that result from any ischemia or ischemia-like condition, particularly a decline in age-related behavioral changes associated with transient ischemic attacks and vascular conditions that mimic ischemia-like conditions. Preferably, the age-related behavioral changes are one or more of forgetfulness, disorientation, changes in sleep and wake habits, loss of “housetraining”, confusion, frustration, or change in temperament.

In a further aspect, the invention provides methods for facilitating, maintaining, or enhancing cognitive function in an animal. The methods comprise administering wolfberry to the animal in an amount effective for facilitating, maintaining, or enhancing cognitive function in the animal. The invention is useful for facilitating, maintaining, and enhancing one or more of learning, attention, memory, concentrating, planning, reasoning, decision-making, spatial orientation, communicating, following instructions, carrying out daily activities, and the like. In a preferred embodiment, the cognitive function is learning. In another, the preferred cognitive function is memory.

In another aspect, the invention provides methods for facilitating, maintaining, or enhancing optimal brain function in an animal. The methods comprise administering wolfberry to the animal in an amount effective for facilitating, maintaining, or enhancing optimal brain function in the animal.

In another aspect, the invention provides methods for reducing memory loss in an animal. The methods comprise administering wolfberry to the animal in an amount effective for reducing memory loss in the animal.

In one aspect, the invention provides methods for increasing trainability in an animal. The methods comprise administering wolfberry to the animal in an amount effective for increasing trainability loss in the animal. For example, administering wolfberry while “potty training” an animal permits the animal to learn the task more quickly than if the training occurred without using wolfberry. Similarly, training a dog or cat to obey verbal, signal, or other commands permits the animal to learn the task more quickly than if the training occurred without administering wolfberry. Also, wolfberry is useful for training feral or wild animals such as animals used in a circus or pets raised in the wild.

In one aspect, the invention provides methods for improving the quality of life of an animal. The methods comprise administering wolfberry to the animal in an amount effective for improving the quality of life of the animal.

In another aspect, the invention provides methods for promoting the health and wellness of an animal. The methods comprise administering wolfberry to the animal in an amount effective for promoting the health and wellness of the animal.

In a further aspect, the invention provides methods for preventing, reducing, or treating damage caused by transient ischemic attacks in an animal. The methods comprise administering wolfberry to the animal in an amount effective for preventing, reducing, or treating damage caused by transient ischemic attacks in the animal.

In another aspect, the invention provides methods for retarding brain aging in an animal. The methods comprise administering wolfberry to the animal in an amount effective for retarding brain aging in the animal. The method is useful for retarding brain aging in animals experiencing ischemia-like conditions that cause the brain to age prematurely compared to normal brain aging.

The inventions are based upon the discovery that wolfberry is effective for counteracting tissue damage or dysfunction caused by ischemia and ischemia-like conditions and overcoming various adverse conditions that result from such damage or dysfunction, e.g., brain damage, impaired cognitive function, impeded learning and memory, reduced social interaction, decreased trainability, reduced brain function, memory loss, brain aging, and strokes. The inventions are useful for preventing damage or injury caused by ischemia and for promoting the health and wellness and increasing the quality of life of animals. While wolfberry is useful for any animal of any age, wolfberry is particularly useful for aging animals that are susceptible to or suffering from damage or dysfunction caused by ischemia, ischemia-like conditions, strokes, and transient ischemic attacks.

Wolfberry is administered to an animal in amounts of from about 0.1 to about 3000 mg/kg/day, preferably from about 10 to about 2000 mg/kg/day, most preferably from about 50 to about 1000 mg/kg/day.

Wolfberry can be administered to the animal in any suitable form using any suitable administration route. For example, wolfberry can be administered in a wolfberry composition, as a wolfberry extract, in a food composition, in a dietary supplement, in a pharmaceutical composition, in a nutraceutical composition, or as a medicament. Similarly, wolfberry can be administered using a variety of administration routes, including oral, intranasal, intravenous, intramuscular, intragastric, transpyloric, subcutaneous, rectal, and the like. Preferably, wolfberry is administered to an animal orally. Most preferably, wolfberry is administered orally to an animal as a dietary supplement or as an ingredient in a food composition.

In a preferred embodiment, wolfberry is administered to an animal as an ingredient in a food composition suitable for consumption by an animal, including humans and companion animals such as dogs and cats. Such compositions include complete foods intended to supply the necessary dietary requirements for an animal or food supplements such as animal treats.

In various embodiments, food compositions such as pet food compositions or pet treat compositions comprise from about 5% to about 50% crude protein. The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include pork, lamb, equine, poultry, fish, and mixtures thereof.

The food compositions may further comprise from about 5% to about 40% fat. Examples of suitable fats include animal fats and vegetable fats. Preferably the fat source is an animal fat source such as tallow. Vegetable oils such as corn oil, sunflower oil, safflower oil, rape seed oil, soy bean oil, olive oil and other oils rich in monounsaturated and polyunsaturated fatty acids, may also be used.

The food compositions may further comprise from about 10% to about 60% carbohydrate. Examples of suitable carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products.

The moisture content for such food compositions varies depending on the nature of the food composition. The food compositions may be dry compositions (e.g., kibble), semi-moist compositions, wet compositions, or any mixture thereof. In a preferred embodiment, the composition is a complete and nutritionally balanced pet food. In this embodiment, the pet food may be a “wet food”, “dry food”, or food of “intermediate moisture” content. “Wet food” describes pet food that is typically sold in cans or foil bags and has a moisture content typically in the range of about 70% to about 90%. “Dry food” describes pet food that is of a similar composition to wet food but contains a limited moisture content typically in the range of about 5% to about 15% or 20% (typically in the form or small biscuit-like kibbles). In one preferred embodiment, the compositions have moisture content from about 5% to about 20%. Dry food products include a variety of foods of various moisture contents, such that they are relatively shelf-stable and resistant to microbial or fungal deterioration or contamination. Also preferred are dry food compositions that are extruded food products such as pet foods or snack foods for either humans or companion animals.

The food compositions may also comprise one or more fiber sources. The term “fiber” includes all sources of “bulk” in the food whether digestible or indigestible, soluble or insoluble, fermentable or nonfermentable. Preferred fibers are from plant sources such as marine plants but microbial sources of fiber may also be used. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, pectin, fructooligosaccharide, short chain oligofructose, mannanoligofructose, soy fiber, arabinogalactan, galactooligosaccharide, arabinoxylan, or mixtures thereof.

Alternatively, the fiber source can be a fermentable fiber. Fermentable fiber has previously been described to provide a benefit to the immune system of a companion animal. Fermentable fiber or other compositions known to skilled artisans that provide a prebiotic to enhance the growth of probiotics within the intestine may also be incorporated into the composition to aid in the enhancement of the benefit provided by the invention to the immune system of an animal.

In some embodiments, the ash content of the food composition ranges from less than 1% to about 15%, preferably from about 5% to about 10%.

In a preferred embodiment, the composition is a food composition comprising wolfberry and from about 15% to about 50% protein, from about 5% to about 40% fat, from about 5% to about 10% ash content, and having a moisture content of about 5% to about 20%. In other embodiments, the food composition further comprises prebiotics or probiotics as described herein.

When administered in a food composition, wolfberry comprises from about 0.1 to about 40% of the food composition, preferably from about 3 to about 30%, more preferably from about 5 to about 20%. In various embodiments, food compositions comprise about 1%, 2%, 4%, 6%, 8%, 10%, 12%, 14%, 16%, 18%, 20%, 22%, 24%, 26%, 28%, 30%, 32%, 34%, 36%, 38%, or 40%.

In another embodiment, wolfberry is administered to an animal in a dietary supplement. The dietary supplement can have any suitable form such as a gravy, drinking water, beverage, yogurt, powder, granule, paste, suspension, chew, morsel, treat, snack, pellet, pill, capsule, tablet, sachet, or any other suitable delivery form. The dietary supplement can comprise wolfberry and optional compounds such as vitamins, preservatives, probiotics, prebiotics, and antioxidants. This permits the supplement to be administered to the animal in small amounts, or in the alternative, can be diluted before administration to an animal. The dietary supplement may require admixing with a food composition or with water or other diluent prior to administration to the animal. When administered in a dietary supplement, wolfberry comprises from about 0.1 to about 90% of the supplement, preferably from about 3 to about 70%, more preferably from about 5 to about 60%.

In another embodiment, wolfberry is administered to an animal in a pharmaceutical or nutraceutical composition. The pharmaceutical composition comprises wolfberry and one or more pharmaceutically or nutraceutically acceptable carriers, diluents, or excipients. Generally, pharmaceutical compositions are prepared by admixing a compound or composition with excipients, buffers, binders, plasticizers, colorants, diluents, compressing agents, lubricants, flavorants, moistening agents, and the like, including other ingredients known to skilled artisans to be useful for producing pharmaceuticals and formulating compositions that are suitable for administration to an animal as pharmaceuticals. When administered in a pharmaceutical or nutraceutical composition, wolfberry comprises from about 0.1 to about 90% of the composition, preferably from about 3 to about 70%, more preferably from about 5 to about 60%.

Wolfberry can be administered to the animal on an as-needed, on an as-desired basis, or on a regular basis. A goal of administration on a regular basis is to provide the animal with a regular and consistent dose of wolfberry or the direct or indirect metabolites that result from such ingestion. Such regular and consistent dosing will tend to create constant blood levels of wolfberry and its direct or indirect metabolites. Thus, administration on a regular basis can be once monthly, once weekly, once daily, or more than once daily. Similarly, administration can be every other day, week, or month, every third day, week, or month, every fourth day, week, or month, and the like. Administration can be multiple times per day. When utilized as a supplement to ordinary dietetic requirements, wolfberry may be administered directly to the animal, e.g., orally or otherwise. Wolfberry can alternatively be contacted with, or admixed with, daily feed or food, including a fluid, such as drinking water, or an intravenous connection for an animal that is receiving such treatment. Administration can also be carried out as part of a dietary regimen for an animal. For example, a dietary regimen may comprise causing the regular ingestion by the animal of wolfberry in an amount effective to accomplish the methods of the invention.

According to the methods of the invention, wolfberry administration, including administration as part of a dietary regimen, can span a period of time ranging from parturition through the adult life of the animal. In various embodiments, the animal is a human or companion animal such as a dog or cat. In certain embodiments, the animal is a young or growing animal. In more preferred embodiments, the animal is an aging animal. In other embodiments administration begins, for example, on a regular or extended regular basis, when the animal has reached more than about 30%, 40%, or 50% of its projected or anticipated lifespan. In some embodiments, the animal has attained 40, 45, or 50% of its anticipated lifespan. In yet other embodiments, the animal is older having reached 60, 66, 70, 75, or 80% of its likely lifespan. A determination of lifespan may be based on actuarial tables, calculations, estimates, or the like, and may consider past, present, and future influences or factors that are known to positively or negatively affect lifespan. Consideration of species, gender, size, genetic factors, environmental factors and stressors, present and past health status, past and present nutritional status, stressors, and the like may also influence or be taken into consideration when determining lifespan.

Wolfberry is administered to an animal for a time required to accomplish one or more objectives of the invention, e.g., preventing, reducing, or treating damage caused by ischemia and ischemia-like conditions; preventing, reducing, or treating strokes and damage caused by strokes; preventing, reducing, or treating alterations in brain function; preventing, reducing, or treating cognitive dysfunction syndrome; preventing, reducing, or treating mild cognitive impairment; preventing, reducing, or treating dementia; preventing, reducing, or treating a decline in social interaction; preventing, reducing, or treating age-related behavioral changes; facilitating, maintaining, and enhancing cognitive function; facilitating, maintaining, and enhancing learning and memory; facilitating, maintaining, and enhancing optimal brain function; reducing memory loss; increasing trainability; improving the quality of life; and promoting the health and wellness in an animal. Preferably, wolfberry is administered to an animal on a regular basis.

In another aspect, the invention provides compositions comprising wolfberry in an amount effective for one or more of preventing, reducing, or treating damage caused by ischemia and ischemia-like conditions; preventing, reducing, or treating strokes and damage caused by strokes; preventing, reducing, or treating alterations in brain function; preventing, reducing, or treating cognitive dysfunction syndrome; preventing, reducing, or treating mild cognitive impairment; preventing, reducing, or treating dementia; preventing, reducing, or treating a decline in social interaction; preventing, reducing, or treating age-related behavioral changes; facilitating, maintaining, and enhancing cognitive function; facilitating, maintaining, and enhancing learning and memory; facilitating, maintaining, and enhancing optimal brain function; reducing memory loss; increasing trainability; improving the quality of life; and promoting the health and wellness in an animal. The compositions contain wolfberry in amounts sufficient to administer wolfberry to an animal in amounts of from about 0.1 to about 3000 mg/kg/day, preferably from about 10 to about 2000 mg/kg/day, most preferably from about 50 to about 1000 mg/kg/day when the compositions are administered as anticipated or recommended for a particular composition. Typically, wolfberry comprises from about 1 to about 90% of a composition, preferably from about 3 to about 70%, more preferably from about 5 to about 60%. In various embodiments, food compositions comprise about 1%, 2%, 4%, 6%, 8%, 10%, 12%, 14%, 16%, 18%, 20%, 22%, 24%, 26%, 28%, 30%, 32%, 34%, 36%, 38%, 40%, 45%, 50%, 55%, 60%, 70%, or 80%.

Wolfberry compositions such as food, dietary, pharmaceutical, and other compositions may further comprise one or more substances such as vitamins, minerals, probiotics, prebiotics, salts, and functional additives such as palatants, colorants, emulsifiers, and antimicrobial or other preservatives. Minerals that may be useful in such compositions include, for example, calcium, phosphorous, potassium, sodium, iron, chloride, boron, copper, zinc, magnesium, manganese, iodine, selenium, and the like. Examples of additional vitamins useful herein include such fat soluble vitamins as A, D, E, and K. Inulin, amino acids, enzymes, coenzymes, and the like may be useful to include in various embodiments.

In certain embodiments, wolfberry compositions further comprise prebiotics or probiotics. Probiotics are live microorganisms that have a beneficial effect in the prevention and treatment of specific medical conditions when ingested. Probiotics are believed to exert biological effects through a phenomenon known as colonization resistance. The probiotics facilitate a process whereby the indigenous anaerobic flora limits the concentration of potentially harmful (mostly aerobic) bacteria in the digestive tract. Other modes of action, such as supplying enzymes or influencing enzyme activity in the gastrointestinal tract, may also account for some of the other functions that have been attributed to probiotics. Prebiotics are nondigestible food ingredients that beneficially affect host health by selectively stimulating the growth and/or activity of bacteria in the colon. Prebiotics include fructooligosaccharides (FOS), xylooligosaccharides (XOS), galactooligosaccharides (GOS), and mannooligosaccharides (typically for non-human foods such as petfoods). The prebiotic, fructooligosaccharide (FOS) is found naturally in many foods such as wheat, onions, bananas, honey, garlic, and leeks. FOS can also be isolated from chicory root or synthesized enzymatically from sucrose. FOS fermentation in the colon results in a large number of physiologic effects including increasing the numbers of bifidobacteria in the colon, increasing calcium absorption, increasing fecal weight, shortening of gastrointestinal transit time, and possibly lowering blood lipid levels. The increase in bifidobacteria has been assumed to benefit human health by producing compounds to inhibit potential pathogens, by reducing blood ammonia levels, and by producing vitamins and digestive enzymes. Probiotic bacteria such as Lactobacilli or Bifidobacteria are believed to positively affect the immune response by improving the intestinal microbial balance leading to enhanced antibody production and phagocytic (devouring or killing) activity of white blood cells. Bifidobacterium lactis could be an effective probiotic dietary supplement for enhancing some aspects of cellular immunity in the elderly. Probiotics enhance systemic cellular immune responses and may be useful as a dietary supplement to boost natural immunity in otherwise healthy adults. Probiotics include many types of bacteria but generally are selected from four genera of bacteria: Lactobacilllus acidophillus, Bifidobacteria, Lactococcus, and Pediococcus. Beneficial species include Enterococcus and Saccharomyces species, e.g., Enterococcus faecium SF68. The amount of probiotics and prebiotics to be administered to the animal is determined by the skilled artisan based upon the type and nature of the prebiotic and probiotic and the type and nature of the animal, e.g., the age, weight, general health, sex, extent of microbial depletion, presence of harmful bacteria, and diet of the animal. Generally, probiotics are administered to the animal in amounts of from about one to about twenty billion colony-forming units (CFUs) per day for the healthy maintenance of intestinal microflora, preferably from about 5 billion to about 10 billion live bacteria per day. Generally, prebiotics are administered in amounts sufficient to positively stimulate the healthy microflora in the gut and cause these “good” bacteria to reproduce. Typical amounts are from about one to about 10 grams per serving or from about 5% to about 40% of the recommended daily dietary fiber for an animal. The probiotics and prebiotics can be made part of the composition by any suitable means. Generally, the agents are mixed with the composition or applied to the surface of the composition, e.g., by sprinkling or spraying. When the agents are part of a kit, the agents can be admixed with other materials or in their own package.

A skilled artisan can determine the appropriate amount of wolfberry, food ingredients, vitamins, minerals, probiotics, prebiotics, antioxidants, or other ingredients to be use to make a particular composition to be administered to a particular animal. Such artisan can consider the animal's species, age, size, weight, health, and the like in determining how best to formulate a particular composition comprising wolfberry and other ingredients. Other factors that may be considered include the type of composition (e.g., pet food composition versus dietary supplement), the desired dosage of each component, the average consumption of specific types of compositions by different animals (e.g., based on species, body weight, activity/energy demands, and the like), and the manufacturing requirements for the composition.

In a further aspect, the invention provides kits suitable for administering wolfberry to an animal. The kits comprise in separate containers in a single package or in separate containers in a virtual package, as appropriate for the kit component, wolfberry and one or more of (1) one or more ingredients suitable for consumption by an animal, (2) one or more probiotics or prebiotics, (3) instructions for how to combine wolfberry and other kit components to produce a composition useful for preventing, reducing, or treating damage caused by ischemia and ischemia-like conditions in animals, (4) instructions for how to use wolfberry and other components of the invention, particularly for the benefit of the animal, (5) a device for preparing or combining the kit components to produce a composition for administration to an animal such as a spoon or other application device, and (6) a device for administering the combined or prepared kit components to an animal such as a bowl or other container.

When the kit comprises a virtual package, the kit is limited to instructions in a virtual environment in combination with one or more physical kit components. The kit contains wolfberry and other components in amounts sufficient to prevent or reduce damage caused by transient ischemic attacks in animals. Typically, wolfberry and the other suitable kit components are admixed just prior to consumption by an animal. The kits may contain the kit components in any of various combinations and/or mixtures. In one embodiment, the kit contains a packet containing wolfberry and a container of food for consumption by an animal. The kit may contain additional items such as a device for mixing wolfberry and other ingredients or a device for containing the admixture, e.g., a food bowl. In another embodiment, wolfberry is mixed with additional nutritional supplements such as vitamins and minerals that promote good health in an animal. The components are each provided in separate containers in a single package or in mixtures of various components in different packages. In preferred embodiments, the kits comprise wolfberry and one or more other ingredients suitable for consumption by an animal. Preferably such kits comprise instructions describing how to combine wolfberry with the other ingredients to form a food composition for consumption by the animal, generally by mixing wolfberry with the other ingredients or by applying wolfberry to the other ingredients, e.g. by sprinkling wolfberry on a food composition.

In another aspect, the invention provides a means for communicating information about or instructions for one or more of (1) using wolfberry for one or more of preventing, reducing, or treating damage caused by ischemia and ischemia-like conditions; preventing, reducing, or treating strokes and damage caused by strokes; preventing, reducing, or treating alterations in brain function; preventing, reducing, or treating cognitive dysfunction syndrome; preventing, reducing, or treating mild cognitive impairment; preventing, reducing, or treating dementia; preventing, reducing, or treating a decline in social interaction; preventing, reducing, or treating age-related behavioral changes; facilitating, maintaining, and enhancing cognitive function; facilitating, maintaining, and enhancing learning and memory; facilitating, maintaining, and enhancing optimal brain function; reducing memory loss; increasing trainability; improving the quality of life; and promoting the health and wellness in an animal; (2) admixing wolfberry and other ingredients to produce a wolfberry composition such as a wolfberry food composition or wolfberry dietary supplement suitable for consumption by an animal; (3) using the kits of the invention for the benefit of an animal, e.g., preventing, reducing, or treating damage caused by ischemia and ischemia-like conditions; preventing, reducing, or treating strokes and damage caused by strokes; preventing, reducing, or treating alterations in brain function; preventing, reducing, or treating cognitive dysfunction syndrome; preventing, reducing, or treating mild cognitive impairment; preventing, reducing, or treating dementia; preventing, reducing, or treating a decline in social interaction; preventing, reducing, or treating age-related behavioral changes; facilitating, maintaining, and enhancing cognitive function; facilitating, maintaining, and enhancing learning and memory; facilitating, maintaining, and enhancing optimal brain function; reducing memory loss; increasing trainability; improving the quality of life; and promoting the health and wellness in an animal; and (4) administering wolfberry to an animal. The means comprises one or more of a physical or electronic document, digital storage media, optical storage media, audio presentation, audiovisual display, or visual display containing the information or instructions. Preferably, the means is selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

In another aspect, the invention provides methods for manufacturing a food composition comprising wolfberry and one or more other ingredients suitable for consumption by an animal, e.g., one or more of protein, fat, carbohydrate, fiber, vitamins, minerals, probiotics, prebiotics, and the like. The methods comprise admixing one or more ingredients suitable for consumption by an animal with wolfberry. Alternatively, the methods comprise applying wolfberry alone or in conjunction or combination with other ingredients onto the food composition, e.g., as a coating or topping. Wolfberry can be added at any time during the manufacture and/or processing of the food composition. The composition can be made according to any method suitable in the art.

In another aspect, the invention provides a package comprising wolfberry and a label affixed to the package containing a word or words, picture, design, acronym, slogan, phrase, or other device, or combination thereof that indicates that the contents of the package contains an ingredient or composition suitable for one or more of preventing, reducing, or treating damage caused by ischemia and ischemia-like conditions; preventing, reducing, or treating strokes and damage caused by strokes; preventing, reducing, or treating alterations in brain function; preventing, reducing, or treating cognitive dysfunction syndrome; preventing, reducing, or treating mild cognitive impairment; preventing, reducing, or treating dementia; preventing, reducing, or treating a decline in social interaction; preventing, reducing, or treating age-related behavioral changes; facilitating, maintaining, and enhancing cognitive function; facilitating, maintaining, and enhancing learning and memory; facilitating, maintaining, and enhancing optimal brain function; reducing memory loss; increasing trainability; improving the quality of life; and promoting the health and wellness in an animal. Typically, such device comprises the words “improves quality of life”, “promotes health and wellness”, “improves cognitive function”, “improves memory”, “reduces memory loss in aging animals”, or an equivalent expression printed on the package. Any package or packaging material suitable for containing the composition is useful in the invention, e.g., a sachet, bag, box, bottle, can, pouch, and the like manufactured from paper, plastic, foil, metal, and the like. In a preferred embodiment, the package contains a food composition adapted for a particular animal such as a human, canine, or feline, as appropriate for the label, preferably a companion animal food composition for dogs or cats. In a preferred embodiment, the package is a sachet comprising wolfberry.

In another aspect, the invention provides for use of wolfberry to prepare a medicament for one or more of preventing, reducing, or treating damage caused by ischemia and ischemia-like conditions; preventing, reducing, or treating strokes and damage caused by strokes; preventing, reducing, or treating alterations in brain function; preventing, reducing, or treating cognitive dysfunction syndrome; preventing, reducing, or treating mild cognitive impairment; preventing, reducing, or treating dementia; preventing, reducing, or treating a decline in social interaction; preventing, reducing, or treating age-related behavioral changes; facilitating, maintaining, and enhancing cognitive function; facilitating, maintaining, and enhancing learning and memory; facilitating, maintaining, and enhancing optimal brain function; reducing memory loss; increasing trainability; improving the quality of life; and promoting the health and wellness in an animal. Generally, medicaments are prepared by admixing a compound or composition, i.e., wolfberry or a wolfberry composition, with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to an animal.

In another aspect, the invention provides methods for extending the prime years of an animal's life. The methods comprise administering a composition comprising wolfberry to the animal in an amount effective for extending the prime for the animal.

### EXAMPLES

The invention can be further illustrated by the following example, although it will be understood that this example is included merely for purposes of illustration and is not intended to limit the scope of the invention unless otherwise specifically indicated.

### Example 1

Two to three month old female Sprague Dawley (SD) rats were purchased from Charles Rivers (Wilmington, Mass.), and maintained in an animal facility in a temperature-controlled room (22-25° C.) with 12-hour dark-light cycles. All rats had free access to laboratory chow and tap water during the acclamation period and to the control or test diet during the 4-week study period. E2 pellets (4 mg/ml in corn oil in 30 mm Silastic pellets) were implanted in the E2 group rats one week before middle cerebral artery (MCA) occlusion (MCAO). During the 4-week feeding period before the MCAO procedure, rats in the control and estradiol (E2) groups were fed a control diet. Rats in the wolfberry extract group were fed the control diet supplemented with 12.5% wolfberry extract.

All animals received ovariectomy (OVX) at least 14 days before any further procedure. For middle cerebral artery (MCA) occlusion (MCAO) and reperfusion, an intraluminal filament model was used. Briefly, the animals were anesthetized with ketomine (60 mg/kg) and xylazine (10 mg/kg), then the internal carotid artery (ICA) was exposed, and a 3-0 monofilament nylon suture was introduced into the ICA lumen through a puncture and gently advanced to the distal internal carotid artery (ICA) until proper resistance was felt. After 1 hr, the suture was withdrawn from the ICA and the distal ICA was immediately cauterized. At 24 hours after the onset of reperfusion, animals were sampled for TTC staining as described below.

Brains were removed and dissected coronally into 2 mm sections using a metallic brain matrix (ASI Instruments, Inc., Warren, Mich.), and stained by incubation in a 2% 2,3,5-Triphenyl-2H-tetrazolium chloride (TTC) in a 0.9% saline solution at 37° C. for 30 min. Photographs were taken with Kodak digital camera. Digital images of each brain section are used to calculate lesion volume for the infracted area of the brain. The results are shown in Table 1.

Referring to Table 1, compared with the control rats, 17-beta estradiol and lacto-wolfberry extract significantly reduced ischemia-induced brain lesion by 48% and 60%, respectively. These data show that lacto-wolfberry extract is very effective in protecting brains against ischemia (stroke)-induced damage.

TABLE 1
Effects of Lacto-Wolfberry and 17 Beta-Estradiol on
Stroke-Induced Damage in Rats

| | | 17 β- | |
|---|---|---|---|
| | Control | Estradiol | Lacto-Wolfberry Extract |
| Sample Size | 14 | 15 | 15 |
| Ischemic Lesion Volume | 286.0 | 147.4 | 114.6 |
| (means, mm³) | | | |
| Std. Error | 25.67 | 23.95 | 19.10 |

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims, the invention may be practiced otherwise than as specifically described.

1. A method for one or more of preventing, reducing, or treating damage caused by ischemia and ischemia-like conditions; preventing, reducing, or treating strokes and damage caused by strokes; preventing, reducing, or treating alterations in brain function; preventing, reducing, or treating cognitive dysfunction syndrome; preventing, reducing, or treating mild cognitive impairment; preventing, reducing, or treating dementia; preventing, reducing, or treating a decline in social interaction; preventing, reducing, or treating age-related behavioral changes; facilitating, maintaining, or enhancing cognitive function; facilitating, maintaining, or enhancing learning and memory; facilitating, maintaining, or enhancing optimal brain function; reducing memory loss; increasing trainability; improving the quality of life; and promoting the health and wellness in an animal, the method comprising administering wolfberry to the animal in an amount effective for one or more of preventing, reducing, or treating damage caused by ischemia and ischemia-like conditions; preventing, reducing, or treating strokes and damage caused by strokes; preventing, reducing, or treating alterations in brain function; preventing, reducing, or treating cognitive dysfunction syndrome; preventing, reducing, or treating mild cognitive impairment; preventing, reducing, or treating dementia; preventing, reducing, or treating a decline in social interaction; preventing, reducing, or treating age-related behavioral changes; facilitating, maintaining, or enhancing cognitive function; facilitating, maintaining, or enhancing learning and memory; facilitating, maintaining, or enhancing optimal brain function; reducing memory loss; increasing trainability; improving the quality of life; and promoting the health and wellness in the animal.

2. The method of claim 1 wherein wolfberry is administered in amounts of from about 0.1 to about 3000 mg/kg/day.

3. The method of claim 1 wherein wolfberry is administered to the animal in a food composition or as a dietary supplement.

4. The method of claim 3 wherein the composition is a food composition and wolfberry comprises from about 0.1 to about 40% of the food composition.

5. The method of claim 3 wherein the composition or dietary supplement further comprises one or more prebiotics or probiotics.

6. The method of claim 1 wherein the animal is a human or a companion animal.

7. The method of claim 6 wherein the companion animal is a canine or a feline.

8. The method of claim 1 wherein the animal is an aging animal.

9. A method for preventing, reducing, or treating damage caused by ischemia and ischemia-like conditions in an animal comprising administering wolfberry to the animal in an amount effective for preventing, reducing, or treating damage caused by ischemia and ischemia-like conditions and ischemia-like conditions in the animal.

10. The method of claim 9 wherein wolfberry is administered in amounts of from about 0.1 to about 3000 mg/kg/day.

11. The method of claim 9 wherein wolfberry is administered to the animal in a food composition or as a dietary supplement.

12. The method of claim 11 wherein the composition is a food composition and wolfberry comprises from about 0.1 to about 40% of the food composition.

13. The method of claim 11 wherein the composition or dietary supplement further comprises one or more prebiotics or probiotics.

14. The method of claim 9 wherein the animal is a human or a companion animal.

15. The method of claim 14 wherein the companion animal is a canine or a feline.

16. The method of claim 9 wherein the animal is an aging animal.

17. A kit suitable for administering wolfberry to an animal comprising in separate containers in a single package or in separate containers in a virtual package, as appropriate for the kit component, wolfberry and one or more of (1) one or more ingredients suitable for consumption by an animal, (2) one or more probiotics or prebiotics, (3) instructions for how to combine wolfberry and other kit components to produce a composition useful for preventing, reducing, or treating damage caused by ischemia and ischemia-like conditions in animals, (4) instructions for how to use wolfberry and other components of the invention, particularly for the benefit of an animal, (5) a device for preparing or combining the kit components to produce a composition for administration to an animal, and (6) a device for administering the combined or prepared kit components to an animal.

18. The kit of claim 17 wherein the wolfberry is in a sachet.

19. The kit of claim 17 comprising wolfberry and one or more ingredients suitable for consumption by an animal.

20. The kit of claim 19 further comprising instructions for how to combine wolfberry and the ingredients to produce a composition useful for preventing, reducing, or treating damage caused by ischemia and ischemia-like conditions in animals.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)
